(19) United States
(12) Patent Application Publication
(10) Pub. No.: US 2016/0291010 A1
(43) Pub. Date: Oct. 6, 2016

(54) IMMUNOCHROMATOGRAPHY STRIP SENSOR CAPABLE OF MEASURING BIOMATERIAL CONCENTRATION OVER BROAD CONCENTRATION RANGE

(71) Applicants: GWANGJU INSTITUTE OF SCIENCE AND TECHNOLOGY, Gwangju (KR); INGIBIO, LTD., Buk-gu gwangju (KR)

(72) Inventors: Min Gon KIM, Gwangju (KR); Hyou Arm JOUNG, Gwangju (KR); Young Kyoung OH, Gwangju (KR)

(21) Appl. No.: 15/036,340

(22) PCT Filed: Nov. 11, 2014
(86) PCT No.: PCT/KR2014/010810

§ 371 Date: May 12, 2016

(30) Foreign Application Priority Data
Nov. 12, 2013 (KR) 10-2013-0136906

(51) Int. Cl. G01N 33/558; G01N 33/543; G01N 33/553; G01N 33/577

(52) U.S. Cl. CPC G01N 33/558; G01N 33/577; G01N 33/54346; G01N 33/553; G01N 2333/805; G01N 2333/4737

(57) ABSTRACT

The present invention relates to an immunochromatography strip sensor capable of measuring a biomaterial concentration over a broad concentration range, and a method for measuring a biomaterial concentration over a broad concentration range using the sensor. A detection method using the sensor according to the present invention can accurately measure an antigen concentration over a broad concentration range, and achieve a low cost, rapidity and convenience, and thus the method is suitable for a point of care test (POCT) requiring rapidity and high sensitivity.

### TECHNICAL FIELD

This application claims the benefit of Korean Patent Application No. 10-2013-00136906, filed on Nov. 12, 2013, which is incorporated herein by reference in its entirety.

The present invention relates to an immunochromatography strip sensor capable of measuring a biomaterial concentration over a wide range of concentrations and a method for measuring a biomaterial concentration over a wide range of concentrations using the sensor.

### BACKGROUND ART

C-reactive protein (CRP) is a well-known biomarker for acute inflammation, belongs to the family of pentraxins, and consists of five repeating subunits. It is synthesized by the liver when stimulated by interleukin-1 and interleukin-6. Over the past few decades, the level of plasma CRP has been used in predicting risk of myocardial infarction, cancers, cardiovascular diseases, and diabetes, or utilized in research into establishing antibiotics treatment guidelines (Documents 1-9). The level of CRP in a healthy and normal adult is 0.8 μg/mL on average and the level of CRP rises to 100 mg/L or more when there is an inflammatory response. In the case of conventionally mild inflammation or virus infection, the level of CRP ranges from 10 to 50 μg/mL. In the case of active inflammation or bacterial infection, the level of CRP ranges from 50 to 200 μg/mL. In the case of heavy inflammation or injury, the level of CRP exceeds 200 μg/mL. The Food and Drug Administration (FDA) has recommended that CRP measurement for inflammation be made in a CRP level of 20 to 500 μg/mL and a high sensitivity CRP (hs-CRP) test that is a reliable biomarker for cardiovascular disorders and a potential risk predictor be made in a CRP level of 1 to 10 μg/mL.

In the case of acute inflammation or primary healthcare, for ensuring rapid diagnosis or consecutive monitoring of the CRP level in the body fluid, quantitative measurements need to be performed within measurable range with rapidity, accuracy, convenience and low cost (Documents 10-11). As a rapid and quantitative CRP analysis, an immunoturbidimetric assay using polystyrene beads, an immunonephelometry assay, and a gold nanoparticle agglomeration assay are often employed in the related art. Further, the recent introduction of a CRP test over a wide range of concentrations allows measurement over a wide range of concentrations. For example, in the case of DIAgam XS Cardio NanoGold C-reactive protein (CRP), it is possible to measure the level of CRP ranging from 0.42 μg/mL to 265 μg/mL using a reagent supplied from DIAgam laboratory (Lille, France). The reagent of DIAgam can be used by diluting the reagent and then used in measuring the level of CRP over all concentration ranges of 0.1 μg/mL to 160 μg/mL by an Olympus AU640 biochemical analyzer. These methods provide accurate values through automated analyzers, but require expensive equipment and skilled experts in order to conduct the analysis processes. In order to overcome these limitations, methods for measuring CRP at low cost, short assay time and simple operation technique suitable for point of care testing (POCT) have been suggested. Among point of care tests with rapid and high sensitivity, microfluidic chips have been broadly studied as a tool for measuring CRP. According to a study by Gervais et al. (Document 12), microfluidic chips could detect CRP in a concentration of 10 ng/mL for 3 minutes and CRP in a concentration of not more than 1 ng/mL for 14 minutes. In a study performed by Jonsson et al. (Document 13), lateral flow polymer chips had a dynamic range of 102 dB and a limit of detection (LOD) of 2.6 ng/mL. Further, in a report by Gervais et al. (Document 12), it was observed that when CRP concentration increased within the range of 0.01 μg/mL to 1.0 μg/mL, the corresponding fluorescence signal intensity decreased. As can be seen from the above results, microfluidic chips can allow very rapid and low concentration measurement. However, microfluidic chips have limitations in view of measurement of high CRP concentrations, mass production and relative cost (Documents 14-15). In order to overcome these limitations, a vertical flow assay (VFA) for CRP analysis was developed (Document 16). In this study, although a dynamic range broader than that of a lateral flow assay (LFA) strip biosensor was used for 1 minute, a wider range of CRP level was not covered

An LFA strip biosensor is fast, low cost and one of single-step immunoassays (Document 17). Though a number of types of LFA strip biosensor based on CRP test strips are commercially available, those LFA strip biosensors have a disadvantage of being incapable of covering the level of serum CRP due to a hook effect that generates false negative results resulting from a very high concentration of an analyte in a sandwich immunoassay (Documents 18-19). For this reason, most commercial LFA strip sensors are employed in high sensitivity CRP (hsCRP) or heart CRP (cCRP) diagnosis. However, in the research by Leung et al. (Document 20), a “digital-style” assay or a “bar code-style” assay using gold nanoparticles (AuNPs) as an indicator provides a semi-quantitative lateral flow assay by counting the number of red lines in the test within 15 minutes, detecting CRP of low concentration, and predicting risks of initial cardiovascular disease (CVD) or bacterial and viral infection. Although these LFA sensors are used for different detection objects depending upon diseases to be detected, these LFA sensors detect the same CRP using a multi-line measurement method. There is a need for a single-step immunosensor for CRP assay over a wide range of concentrations ranging from 0 to 500 mg/L. Further, LFA biosensors have important elements for evaluating rapid diagnostic sensors or quality of point-of-care test such as convenience, commercial availability and manufacturing cost (Document 21).

A number of articles and patent documents are cited throughout the specification. The cited articles and patent documents are incorporated herein by reference for description of the technical field of the present invention and for clear description of the present invention.

1. Triant V A, Meigs J B, Grinspoon S K. Journal of acquired immune deficiency syndromes 2009; 51:268-73.

2. Patrick L, Uzick M. Journal of clinical therapeutics 2001; 6: 248-71.

3. Ridker P M. Circulation 2003; 107: 363-9.

4. Kushner I, Broder M L, Karp D. The Journal of clinical investigation 1978; 61:235-42.

5. Ridker P M. Cardiology patient page, Circulation 2003; 108: e81-5.

6. Dufour J F, Hepatology 2013; 57: 2103-5.

7. Mazhar D, Ngan S. QJM: monthly journal of the Association of Physicians 2006; 99: 555-9.

8. Kasapis C, Thompson P D. Journal of the American College of Cardiology 2005; 45: 1563-9.

9. Rifai N, Ridker P M. Clinical chemistry 2001; 47: 403-11.

10. Junker R, Schlebusch H, Luppa P B. Dutsches Arzteblatt international 2010; 107: 561-7.

11. Pfaffin A, Schleicher E. Analytical and bioanalytical chemistry 2009; 393: 1473-80.

12. Gervais L, Delamarche E. T. Lab on a chip 2009; 9: 3330-7.

13. Jonsson C, Aronsson M, Rundstrom G, Pettersson C, Mendel-Hartvig I, Bakker J, et al., Lab on a chip 2008; 8: 1191-7.

14. Revet C, Lee H, Hirsche A, Hamilton S, Lu H. Chemical Engineering Science 2011; 66: 1490-507.

15. Yager P, Edwards T, Fu E, Helton K, Nelson K, Tam M R, Weigl B H. Nature 2006; 442: 412-8.

16. Oh Y K, Joung H A, Kim S, Kim M G, Lab Chip 2013; 13: 768-72.

17. Posthuma-Trumpie G A, Korf J, Amerongen Av. Anal Bioanal Chem 2009; 393: 569-82.

18. Fernando S A, Wilson G S, Journal of immunological methods 1992; 151: 47-66.

19. Rodbard D, Feldman Y, Jaffe M L, Miles L E, Immunochemistry 1978; 15: 77-82.

20. Leung W, Chan C P, Rainer T H, Ip M, Cautherlay G W, Renneberg R. Journal of immunological methods 2008; 336-: 30-6.

21. Luong J H, Male K B, Glennon J D. Biotechnology advances 2008; 26: 492-500.

### DISCLOSURE

### Technical Problem

The present inventors have conducted extensive researches to develop a biosensor capable of measuring correct concentrations of biomaterials over a wide range of concentrations. As a result, it was experimentally confirmed that an antigen line may be further added between a control line and a test line in a lateral flow assay strip sensor based on an immunochromatographic method, thereby ensuring rapid and accurate measurement of an antigen concentration over a wide range of concentrations. The present invention has been achieved based on this finding.

Therefore, it is an object of the present invention to provide a lateral flow assay strip sensor for measuring an antigen concentration over a wide range of concentrations.

It is another object of the present invention to provide a method for measuring an antigen concentration over a wide range of concentrations using the lateral flow assay strip sensor.

The above and other objects and advantages of the present invention will be clearly understood by the following detailed description, claims and drawings.

### Technical Solution

In accordance with one aspect of the present invention, there is provided a lateral flow assay strip sensor for measuring an antigen concentration over a wide range of concentrations, wherein the lateral flow assay strip sensor includes a structure in which a sample pad, a conjugate pad, a membrane and an absorption pad are sequentially connected, the conjugate pad is provided with a nanoparticle-detection antibody conjugate composed of nanoparticles and a detection antibody to bind the antigen, the membrane is provided with a test line, an antigen line and a control line formed sequentially in a direction from the conjugate pad to the absorption pad, the test line includes an immobilized capture antibody to bind the antigen, the antigen line includes an immobilized antigen, the control line includes an immobilized secondary antibody to bind the detection antibody.

The lateral flow assay (LFA) strip sensor may include a structure in which a sample pad, a conjugate pad, a membrane and an absorption pad are sequentially connected.

In the sensor according to the present invention, the “sample pad” is intended to refer to a pad capable of receiving a sample to be assayed, allowing divergent flow, and the sample pad is composed of a material having porosity sufficient for receiving and retaining a sample to be assayed. Examples of such a porous material may include fibrous paper; a microporous membrane consisting of cellulose materials, cellulose, a cellulose derivative such as cellulose acetate, nitrocellulose, glass fiber, textiles such as natural cotton and nylon, and porous gels, without being limited thereto.

In the sensor according to the present invention, the “conjugate pad” refers to a pad capable of receiving a sample diffusing and moving from the sample pad while including a nanoparticle-detection antibody conjugate composed of nanoparticles and a detection antibody to bind the antigen. The conjugate pad is composed of a material capable of divergent flow like the sample pad.

The nanoparticles of the “nanoparticle-detection antibody conjugate” included in the conjugate pad means nanoparticles capable of functioning as a detectable label. The nanoparticles are preferably nanoparticles of a metal. Examples of the metal may include noble metals such as gold (Au), silver (Ag), platinum (Pt), palladium (Pd), iridium (Ir), rhodium (Rh), ruthenium (Ru); transition metals such as titanium (Ti), zirconium (Zr), hafnium (Hf), vanadium (V), niobium (Nb), tantalum (Ta), chromium (Cr), molybdenum (Mo), tungsten (W), ruthenium (Ru), osmium (Os); metals such as iron (Fe), nickel (Ni), and cobalt (Co); metal oxides such as magnesium oxide (MgO), titanium dioxide (TiO₂), vanadium pentoxide (V₂0₅), and zinc oxide (ZnO), without being limited thereto. The nanoparticles are most preferably gold (Au) nanoparticles.

The detection antibody means an antibody specifically binding to an antigen to be assayed. The detection antibody includes a fragment of an antibody so long as the detection antibody possesses binding specificity.

The detection antibody may include monoclonal antibodies or polyclonal antibodies. Most preferably, monoclonal antibodies are used.

The bond between the nanoparticles and detection antibody may include, for example, an ion bond, covalent bond, metal bond, coordinated bond, hydrogen bond, and van der Waals bond, without being limited thereto.

In the sensor according to the present invention, the “membrane” may be prepared by using various materials through which sample materials can penetrate. For example, the membrane may be formed from a material selected from the naturally occurring materials, synthetic materials or naturally occurring materials deformed by synthesis, for example, polysaccharides (for instance: cellulose materials, paper, cellulose derivatives such as cellulose acetate and nitrocellulose); polyether sulfone; polyethylene; nylon; polyvinylidene fluoride (PVDF); polyester; polypropylene; silica; inorganic materials uniformly dispersed in a porous polymer matrix together with vinyl chloride, vinyl chloride-propylene copolymers and vinyl chloride-vinyl acetate copolymers, for example inactivated alumina, diatomaceous earth, MgS0₄, or other inorganic fine powder material; naturally occurring (for example: cotton) and synthetic (for example: nylon or rayon) textiles; porous gels, for example, silica gel, agarose, dextran and gelatin; polymer films, for example, polyacrylamide; and the like. Preferably, the membrane is prepared from polymer materials, for example, nitrocellulose, polyethersulfone, polyethylene, nylon, polyvinylidene fluoride, polyester and polypropylene.

In the sensor according to the present invention, the “absorption pad” may be positioned at a distal end of the membrane or adjacent to the distal end of the membrane. The absorption pad generally receives fluidal samples moving through the entire membrane. The absorption pad may facilitate capillary action and diffusive flow through the membrane.

The sensor according to the present invention consists of the sample pad, the conjugate pad, the membrane and the absorption pad sequentially positioned on an identical backing card.

The backing card may be formed from any materials so long as the backing card can support and transport the sample pad, the conjugate pad, the membrane and the absorption pad. In general, it is preferred that the backing card is liquid impervious so that sample fluids diffusing through the membrane are not leaked. Materials for the backing card may include, for example, glass; polymer materials, for example, polystyrene, polypropylene, polyester, polybutadiene, polyvinyl chloride, polyamide, polycarbonate, epoxide, methacrylate, and polymelamine, without being limited thereto.

In the sensor according to the present invention, a test line, an antigen line and a control line are sequentially formed on the membrane in a direction from the conjugate pad to the absorption pad.

In the sensor according to the present invention, the “test line” includes an immobilized capture antibody to bind the antigen.

In the sensor according to the present invention, the “antigen line” includes an immobilized antigen.

Preferably, the antigen at the antigen line is immobilized via the capture antibody immobilized on the membrane Immobilizing the antigen at the antigen line through binding to the capture antibody rather than immobilizing the antigen directly on the membrane can allow the antigen to be exposed in a suitable direction so that the antigen easily binds to the nanoparticle-detection antibody, thereby enhancing performance of the sensor to detect the antigen.

In the sensor according to the present invention, the “control line” includes an immobilized secondary antibody capable of binding the detection antibody. The secondary antibody at the control line binds to a nanoparticle-detection antibody complex or a nanoparticle-detection antibody-antigen complex, and generates detection signals for such bindings.

The term “antigen” as used herein refers to a substance, concentration or presence of which is to be assayed. Examples of antigens may include proteins, peptides, microorganisms, amino acids, nucleic acids, hormones, steroids, vitamins, drugs, bacteria, viruses, and the like, without being limited thereto. It is most preferred that the antigen is C-reactive protein (CRP).

The term “sample” as used herein refers to a biological material that is suspected to contain an antigen to be assayed. Examples of samples may include blood, interstitial fluid, saliva, ocular lens fluid, cerebrospinal fluid, sweat, urine, milk, ascitic fluid, mucus, nasal fluid, hemoptysis, joint fluid, peritoneal fluid, vaginal lubrication, menstrual secretion, amniotic fluid, and sperm. The sample may be originated from any biological sources such as physiological fluids.

In accordance with another aspect of the present invention, there is provided a method for measuring an antigen concentration over a wide range of concentrations using a lateral flow assay strip sensor including a structure in which a sample pad, a conjugate pad, a membrane and an absorption pad are sequentially connected, the conjugate pad being provided with a nanoparticle-detection antibody conjugate composed of nanoparticles and a detection antibody to bind the antigen, the membrane being provided with a test line, an antigen line and a control line formed sequentially in a direction from the conjugate pad to the absorption pad, the test line including an immobilized capture antibody to bind the antigen, the antigen line including an immobilized antigen, and the control line including an immobilized secondary antibody to bind the detection antibody, the method including:

(a) bringing a sample containing an antigen to be assayed in a predetermined concentration into contact with the sample pad;

(b) measuring signals produced from the test line, the antigen line and the control line;

(c) performing steps (a) and (b) using samples containing an antigen to be assayed in a predetermined concentration different from the concentration in step (a);

(d) calculating a calibration curve from signal values measured from steps (a) to (c);

(e) bringing a sample containing an antigen to be assayed in an unknown concentration into contact with the sample pad;

(f) measuring signals produced from the test line, the antigen line and the control line; and

(g) calculating a concentration of the antigen contained in the unknown concentration using the measured signal values and the calibration curve of step (d).

According to exemplary embodiments, in step (g), when comparing the signal values measured at the antigen line with the signal values measured at the control line, the lowest value of the calculated antigen concentrations is chosen in the case where the signal values at the antigen line are larger than the signal values measured at the control line, and the highest value of the calculated antigen concentrations is chosen when the signal values at the antigen line are less than the signal values measured at the control line.

If an antigen concentration over a wide range of concentrations is measured by employing the sensor according to the present invention, signals from the test line exhibit a signal intensity bell curve due to hook effect. Namely, signal intensities at the test line increase as the antigen concentration to be assayed increases. When the antigen concentration is higher than a critical point, signal intensities decrease, thereby forming a bell curve. Therefore, signal intensities produced by one sample measurement may indicate two different concentration results (see panel B in FIG. 5). In order to determine actual concentration values, signal intensities at the control line and the antigen line are compared. Under optimum conditions, signal intensities at the control line are lower than signal intensities at the antigen line when a sample containing a relatively low antigen concentration is used. On the contrary, signal intensities at the antigen line are lower than signal intensities at the control line when a sample containing a relatively high antigen concentration is used. By this phenomenon, it is possible to accurately measure an antigen concentration over a wide range of concentrations in a biological sample.

According to an exemplary embodiment of the present invention, the antigen concentration that can be assayed by the method of the present invention ranges from 1 ng/mL to 500 μg/mL.

### Advantageous Effects

The present invention relates to an immunochromatography strip sensor capable of measuring a biomaterial concentration over a wide range of concentrations and a method for measuring a biomaterial concentration over a wide range of concentrations using the sensor. The detection method using the sensor according to the present invention can accurately measure an antigen concentration over a wide range of concentrations, and achieve low cost, rapidity and convenience, and thus the method is suitable for a point of care test (POCT) requiring high speed and sensitivity.

### DESCRIPTION OF DRAWINGS

FIG. 1 is a schematic view of an LFA strip sensor to which an antigen is introduced, wherein panel A depicts the structure of the LFA strip sensor, panel B depicts principles of detection, and panel C depicts data processing procedures.

FIG. 2 shows comparison results of signal intensity at 1 μg/mL CRP between the case where CRP is directly bound to a pre-treated antibody at an antigen line and the case where peg-CRP is bound to a pre-treated antibody at the antigen line, wherein panel A depicts images of signal intensity and panel B depicts graphs of signal intensity.

FIG. 3 shows comparison results of signal intensity of antigen lines depending upon types of pre-treated antibody.

FIG. 4 shows all data processing procedures.

FIG. 5A shows images of CRP over various concentrations. FIG. 5B shows plotted signal intensity represented in logarithmic unit at three lines, that is, a test line, an antigen line and a control line. FIG. 5C shows results measured after data processing.

FIG. 6 shows test results for 50 clinical samples diluted 10 folds.

FIG. 7 shows test results for myoglobin in the blood serum depending upon concentrations, wherein panel A depicts images and panel B depicts graphs.

FIG. 8 shows assay results after completion of data processing depending upon myoglobin concentrations in logarithmic unit.

### MODE FOR INVENTION

Hereinafter, the present invention will be described in more detail with reference to the following examples. It should be understood that these examples are provided for illustration only and are not to be construed in any way as limiting the present invention.

### EXAMPLE

### Experimental Materials and Methods

1. Experimental Materials

Serums that do not contain CRP (90R-100), surfactant 10G (95R-103), and bovine serum albumin (BSA) were purchased from Fitzgerald Industries International (Acton, Mass., USA). CRP was purchased from Wako Chemicals (309-51191; Osaka, Japan), anti-CRP polyclonal antibody and monoclonal antibody were purchased from Abcam Inc. (Cambridge, Mass., USA). Nitrocellulose membrane was purchased from Millipore Corporation (HFB02404;Billerica, Mass., USA). A sample pad (P/N BSP-133-20) and an absorption pad were purchased from Pall Co. (Port Washington, N.Y., USA). An inter-pad (Fusion8151-6621) was purchased from Whatman (Kent, UK). Each gold colloidal solution was purchased from BB International (EM.GC10, 15, 20, 40; Cardiff, UK). Polyvinyl pyrrolidone (PVP10), sucrose (S7903), sodium azide (S8032) and other compounds were purchased from Sigma-Aldrich (St. Louis, Mo., USA). All buffer solutions and reagent solutions were prepared using purified water by a Milli-Q water purification system. Laminated cards were obtained from Millipore Corporation (HF000MC100).

2. Preparation of AuNP-Antibody Conjugate Solution

In order to prepare an AuNP conjugate, 10 mL of PBS containing an anti-CRP antibody (1 mg/mL concentration) was added to 1 mL of a mixture of 20 nm AuNP colloid and a borate buffer solution (0.1 M, pH 8.5). After incubation at room temperature for 30 minutes, 0.1 mL of PBS containing 10 mg/mL of BSA (bovine serum albumin) was added to the resulting solution in order to block the surface of AuNP. After incubation at 4° C. for 60 minutes, the resulting mixture was subjected to centrifugation at 12,000 rpm and 10° C. for 15 minutes by a HANIL microcentrifuge (micro 17TR; Quasar Instruments, Colorado Springs, Colo., USA). After discarding the supernatant, 10 mM Tris-HCl buffer solution (pH 7.4) was added to the resulting AuNP conjugate to resuspend the conjugate. The centrifugation and suspending procedures were repeated twice, thereby obtaining, as a final suspension, 10 mM Tris-HCl buffer solution containing 0.1% BSA, 0.05% NaN₃ and 1% sucrose was prepared.

3. Preparation of LFA (Lateral Flow Immunoassay) Sensor-1

An LFA sensor is composed of four components, namely, a sample pad, a conjugate pad, a nitrocellulose membrane and an absorption pad. Most components are fixed on a conventional backing card typically composed of an inactive plastic, for example, polyester. A capture antibody and CRP or PEG-CRP or a capture antibody, and a control antibody were immobilized at different regions of an NC membrane (0.25 cm×30 cm) by a dispenser (DCI100; Zeta Corporation, Kyunggi-do, South Korea). At the antigen line, after fixing an antibody (1 mg/mL) in an amount of 1 μL/cm at the cellulose membrane, 1 mg/mL of CRP antigen was sequentially immobilized thereon. A space between each region was set to about 3 mm. The membrane, having passed through loading, was dried in a temperature & humidity chamber at 28° C. for 1 hour. After drying the nitrocellulose (NC) membrane, the membrane was cut into strips with a width of 3.8 mm by a cutter. A concentrated 2× AuNP-antibody conjugate (12) was incubated on the conjugate pad (0.75 cm×0.38 cm), followed by sequentially drying in a temperature & humidity chamber (Hanyang Scientific Equipment Co., Ltd, Korea) at a temperature of 28° C. and humidity of 25%, and then stored. An absorption pad (1.5 cm×0.38 cm) was attached to an upper end of the strip. A sample pad (1.5 cm×0.38 cm) was sequentially assembled on an adhesive plastic backing card (6 cm×0.38 cm) including an NC membrane having an immobilized antibody and an absorption pad. Each segment was overlapped with each other by 1.5 mm or so in order to facilitate solution migration during the assay procedures.

4. Preparation of LFA (Lateral Flow Immunoassay) Sensor-2

A capture antibody, myoglobin antigen, and a control antibody were immobilized at different regions on a nitrocellulose membrane (0.25 cm×30 cm) respectively by a dispenser (DCI100; Zeta Corporation, Kyunggi-do, South Korea). At the antigen line and the test line, a capture antibody (1 mg/mL) was immobilized in an amount of 1 μL/cm at the cellulose membrane. An antigen line was fixed between the two lines. At the antigen line, a capture antibody (1 mg/mL) was immobilized in an amount of 1 μL/cm at the cellulose membrane, followed by sequentially immobilizing a myoglobin antigen (0.5 mg/mL) thereon. Space between each region was set to about 3 mm. The membrane having passed through loading was dried in a temperature & humid chamber at 37° C. for one hour. After drying the nitrocellulose (NC) membrane, the membrane was cut into strips with a width of 3.8 mm by a cutter. A concentrated 3× AuNP-antibody conjugate was incubated on an incubation pad (1 cm×0.38 cm), followed by drying in a temperature & humidity chamber (Hanyang Scientific Equipment Co., Ltd, Korea) at a temperature of 37° C. and humidity of 20%, and then stored. An absorption pad (1.5 cm×0.38 cm) was attached to an upper end of the strip. A sample pad (1.5 cm×0.38 cm) was sequentially assembled on an adhesive plastic backing card (6 cm×0.38 cm) including NC membrane having an immobilized antibody and an absorption pad. Each segment was overlapped with each other by 2 mm or so in order to facilitate solution migration during the assay procedures.

5. Assay of CRP

CRP solutions having various concentrations were prepared using human serums that do not contain CRP. The prepared sample solutions were incubated on an LFA sensor. Images for CRP were obtained using a Chemi Doc™ XRS+ imaging system (Bio-Rad) and color intensity was measured using Image lab™ 4.0 software (Bio-Rad).

6. Assay of Myoglobin

Myoglobin solutions having various concentrations were prepared using human serums that do not contain myoglobin. The prepared sample solutions were incubated on an LFA sensor. Images for myoglobin were obtained using a Chemi Doc™ XRS+ imaging system (Bio-Rad), and color intensity was measured using Image lab™ 4.0 software (Bio-Rad).

7. Preparation of Patient Sample

Serum samples were obtained from 50 patients at Kyungbuk University Hospital. The present inventors explained the research protocols to the patients and received consent for the research. Blood samples were taken in tubes containing lithium heparin anticoagulant (S-Monovette, Numbrecht, Germany) by venipuncture. Thereafter, the blood sample tubes were subjected to centrifugation at 1,000×g for 15 minutes to remove cells in the blood. The concentrations of the harvested blood samples were measured using HITACHI 7180 equipment. The blood plasma was stored at −80° C. until further assay was performed. Samples were diluted 10 fold using serum not containing CRP before measurement.

### Results and Discussion

1. Assay Principle of Strip Sensor

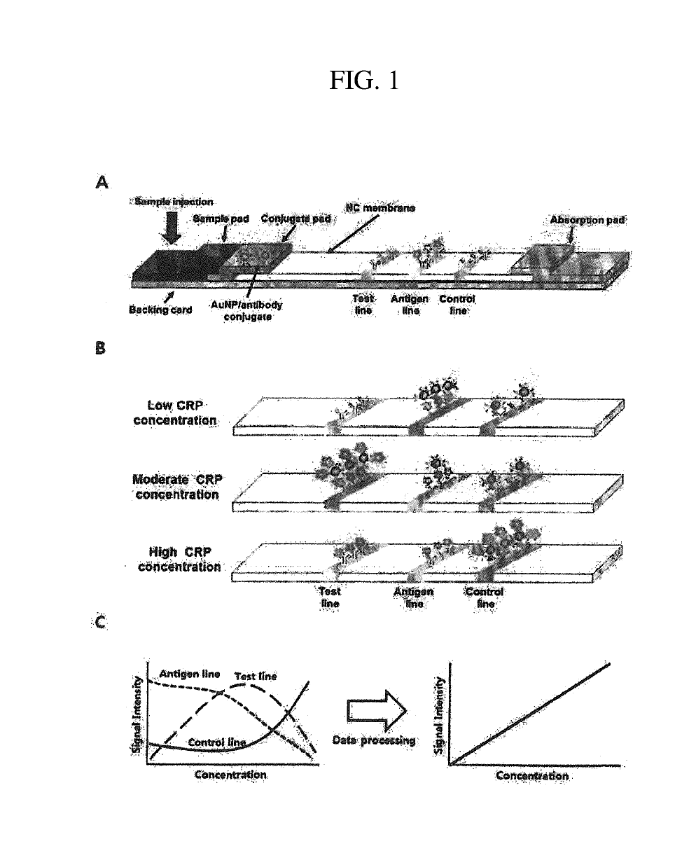

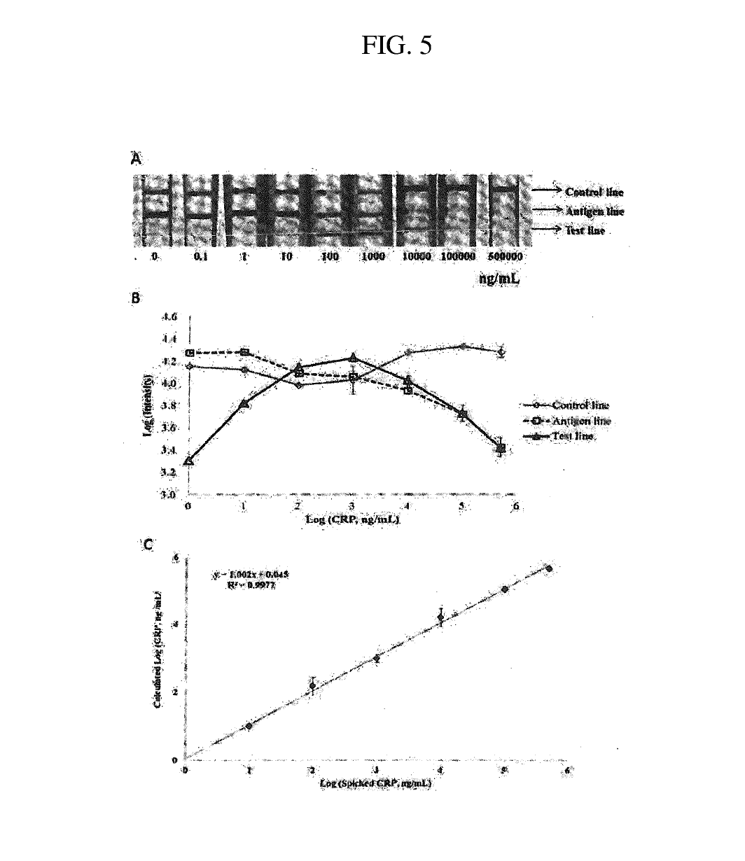

The LFA strip sensor consisted of a test line, an antigen line and a control line sequentially in a sample injection direction (panel A in FIG. 1). When a serum sample solution was incubated on the sample pad, the solution contacted the sample pad, and dampened the sample pad, the conjugation pad and the NC membrane in a transverse direction. At first, at the test line, a sandwich reaction occurred between an antigen and an AuNP-monoclonal antibody conjugate complex for binding to an antibody immobilized at the test line, and AuNP-antibody conjugate that did not react at the test line was bound to CRP antigen immobilized at the antigen line on the NC membrane. In the case of assaying an antigen of a low concentration, the antigen line showed the highest signal intensity, whereas, in the case of assaying an antigen of a high concentration, the antigen line showed the lowest signal intensity or no signal intensity. Further, the control line was employed as a region capable of reacting with a gold labeled monoclonal antibody. The entire reaction is shown in panel B in FIG. 1. Results measured in different antigen concentrations are depicted after passing through data processing procedures (panel C in FIG. 1). The test line at which the injected sample met the AuNP complex for the first time exhibited red signals when the AuNP-antibody conjugate-antigen complex bound to the CRP antigen was bound to an anti-CRP polyclonal antibody immobilized on the NC membrane. As a result, signal intensities at the test line increased in the antigen concentration range of 0 μg/mL to 1 μg/mL and decreased in the antigen with high concentration (panels A and B in FIG. 5). It was assumed that such a phenomenon was caused by a hook effect appearing upon assaying antigens of high concentrations. In the case where antigens are present in a certain concentration or more, antigens that do not bind to AuNP at the conjugate pad rapidly migrate along the NC membrane and bind to a capture antibody present at the test line, since the AuNP antibody conjugate complex having a relatively large size and slow migration speed loses the opportunity to bind the capture antibody at the test line. Due to such a hook effect, signal intensity values of the test line can have a limitation for a wide range of antigen concentrations required in actual measurement in the case of measuring antigens of high concentrations. In order to overcome this phenomenon, an antigen line was introduced so as to use a relationship between the test line, the antigen line and the control line. After injecting samples, the antigen line exhibited different signal values depending upon the concentration of antigen in the samples. For example, the antigen line showed the highest signal value in contrast to the signal exhibited at the test line when the concentration of the antigen was 0. On the contrary, the signal intensity of the antigen line gradually decreased as the concentration of the antigen increased, and the test line showed gradual increase in signal intensity depending upon the concentration of the antigen and showed a tendency of decreasing in signal intensity at an antigen concentration of 1 μg/mL or more. Finally, the two lines showed no red signals at a concentration of 500 μg/mL (panel A in FIG. 5). At the test line, an AuNP conjugate-antibody (Ab)-antigen (Ag) complex bound to a capture antibody in a sandwich assay form, whereas unreacted AuNP conjugate-antibody (Ab) migrated along the NC membrane in an upper direction and bound to an antigen at the antigen line. When an excess of antigen was bound to a capture antibody at the test line prior to formation of the AuNP conjugate-Ab-Ag complex, binding affinity to CRP immobilized on the NC membrane was also decreased. As a result, since immunoassay reaction was inhibited in a high concentration of antigen, no signal intensity was observed at both the test line and the antigen line, and the control line to which anti-mouse IgG antibody was immobilized was bound to an unreacted AuNP-conjugate-antigen complex, thereby exhibiting the highest signal.

2. Optimization of Antigen Line

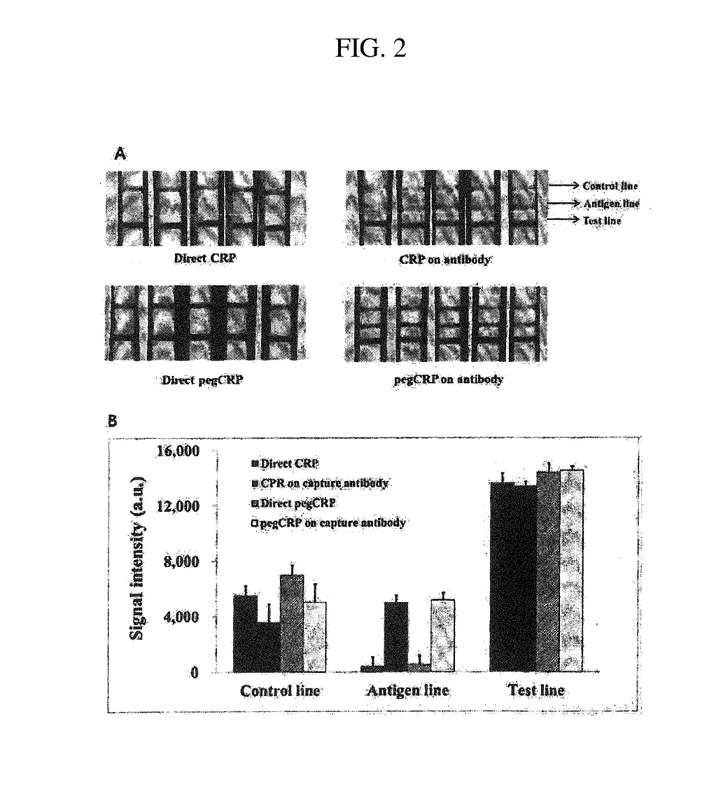

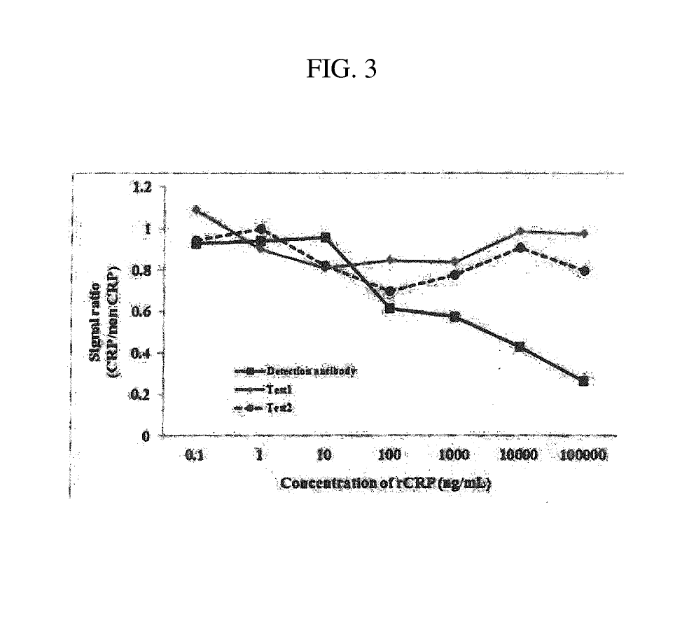

In order to prepare a strip sensor, the most essential factor is to immobilize CRP at the antigen line. The present inventors intended to immobilize a capture antibody at the antigen line in order to assist direction and stabilization of CRP, to which CRP was added. When CRP was directly fixed to an NC membrane, the immobilized antigen and the AuNP conjugate antibody showed no reaction. Postulating that CRP would be degraded during a CRP drying process, CRP was pegylated using NHS-PEG in order to avoid such disadvantages. However, the antigen line to which the pegylated CRP was attached showed no signals (FIG. 2). In an effort to maintain binding affinity between the CRP antigen and AuNP-detection antibody conjugate at the antigen line, a new immobilization method was attempted wherein a capture antibody was previously immobilized and then an antigen was immobilized on the capture antibody. Namely, a capture antibody was loaded on the antigen line of the NC membrane, followed by adding CRP such that CRP was immobilized to the capture antibody, thereby obtaining a strip sensor. The strip sensor thus prepared had no problem in view of CRP binding affinity. In this regard, it was assumed that the affinity was decreased depending upon physical space with the AuNP-detection antibody conjugate and direction of CRP. Further, it was determined that the strip sensor did not affect degradation of proteins during the drying process. In addition, similar signal intensity was observed between the CRP line and pegylated CRP line. Such a phenomenon was not observed when other antibody instead of the capture antibody was used as a previously immobilized antigen. The present inventors performed experiments using two different sorts of antibodies, wherein one showed uniform signal intensity without any change in concentration and the other showed decreased signal intensity until a certain concentration or maintained uniform signal at a concentration higher than the certain concentration (FIG. 3). The antibody used at the antigen line and the test line was a polyclonal antibody, and the polyclonal antibody at the test line and the antigen line was purchased from the same company in order to allow convenience in view of preparation of each line and to facilitate immobilization of the antigen at the antigen line. Further, in order enhance efficiency of competitive reaction at the antigen line, the detection antibody in the AuNP-detection antibody conjugate was a monoclonal antibody. In FIG. 3, a graph for the detection antibody was obtained using a monoclonal antibody as the detection antibody, graphs for test antibody 1 (Test 1) and test antibody 2 (Test 2) were obtained using a polyclonal antibody as the detection antibody. In the case of using polyclonal antibodies as the detection antibody, it was confirmed that, unlike the case where monoclonal antibody was used, there appeared no decrease in signals depending upon CRP concentrations to be measured and uniform signals were maintained.

3. Measurement of CRP Level over a Wide Range of Concentrations and Data Processing

In order to measure CRP over a wide range of concentrations, an antigen concentration was determined by using a hook effect that allows signal intensity to be decreased at a concentration of 1 μg/mL or more at the test line and signals decreasing depending upon the increase in antigen concentration at the antigen line. Theoretically, the shape of the signal intensity curve in single-step sandwich immunoassay is dependent on the concentration of the detection antibody and the capture antibody (Document 18). On the other hand, in the system according to the present invention, the calibration curve in logarithmic unit fits a quadratic equation very closely. For this reason, in order to obtain calibration curves in the shape of a quadratic equation, optimization procedures for antibody concentration or antibody screening test were not performed. After completion of measurement, two different concentrations were obtained from the calibration curve using a quadratic equation, and then actual values were determined by comparing signal intensities between the antigen line and the control line. Namely, the lowest value was chosen when signal values at the antigen line were higher than the signal values at the control line, and the highest value was chosen when signal values at the control line were higher than the signal values at the antigen line.

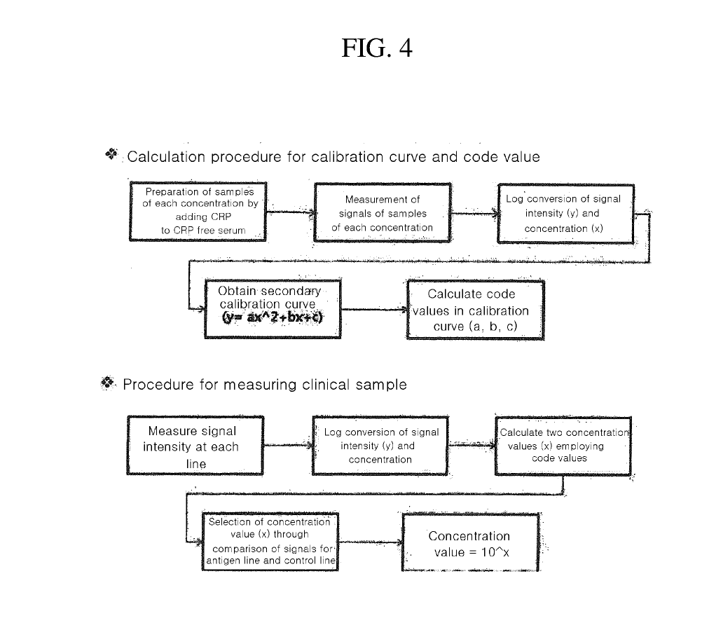

The following detailed description will be given of data processing procedures. Signal intensities at each line on the strip were measured using Chemi_Doc MP equipment. The logarithms of the signal intensities and the concentrations of samples were taken and correction curves for quadratic equations were obtained therefrom using Excel program. The correction curves obtained through Excel program may be represented as: y=ax²+bx+c. From measured correction curves, code values for a, b, and c could be obtained. In the case of unknown samples, signal intensities are measured and the logarithms of the signal intensities were taken in the same manner as above. The values were substituted into y value of the correction curve and code values for a, b, and c were introduced, thereby calculating two different x values. Among these two x values obtained as above, one was chosen in view of correlation with the antigen line and the control line, and actual vales were calculated as a square root of 10. The data processing procedures are depicted in FIG. 4.

Panel C in FIG. 5 shows measured results of CRP over a wide range of concentration through data processing procedures. The experimental results indicate that the sensor according to the present invention can measure the CRP concentration in a level of 10⁵ to 10⁶. In addition, a limit of detection (LOD, blank signal+3 standard deviations) of 0.649 ng/mL was obtained. This value is the highest concentration and indicates that it is possible to develop a sensor capable of measuring CRP concentration of 1.07 mg/mL.

4. Measurement of Clinical Sample

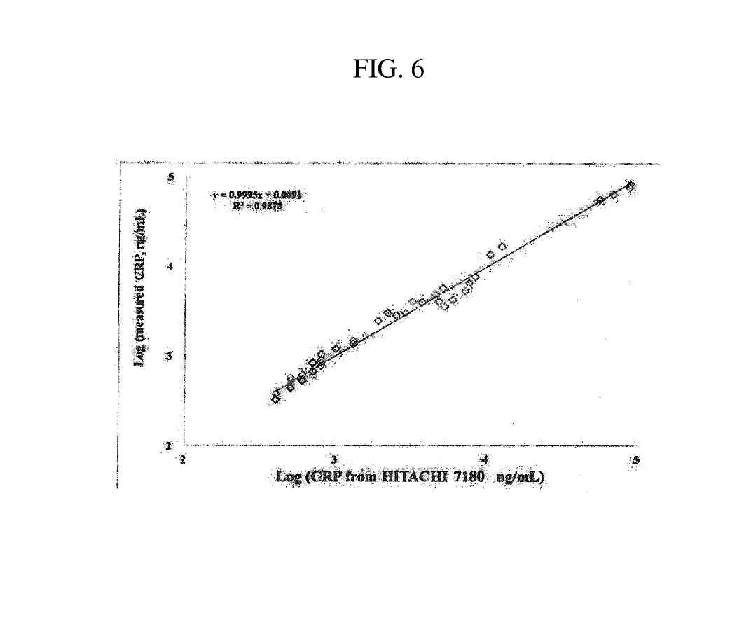

In order to confirm actual application and commercialization possibility, 50 clinical samples were evaluated. Clinical samples were diluted 10 fold with the serum containing no CRP in order to avoid matrix interference, and then measured using the developed strip sensor. As a result, a linear correlation was obtained in samples tested at 0.4 μg/mL to 84.7 μg/mL (FIG. 6). Although the entire CRP concentrations (1 ng/mL to 500 μg/mL) were not estimated in actual sample measurement, it was confirmed that quantitative measurement of CRP level over a wide range of concentrations in human serum can be made through the sensor according to the present invention. It was also confirmed that the antigen line and the assay method of the present invention can be applied to actual sample measurement.

5. Measurement of Myoglobin Antigen

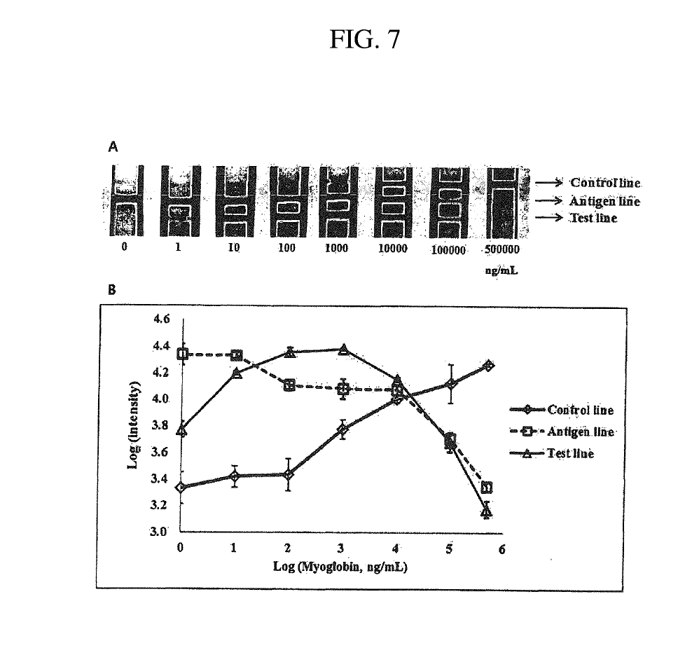

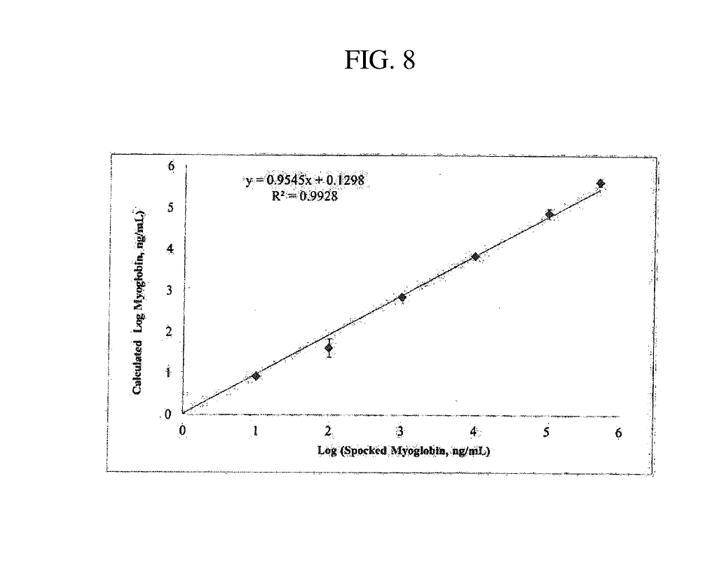

In the method for measuring a wide range of concentrations according to the present invention, in order to prove that the method can be applied to all antigens besides the measured CRP, myoglobin as one of biomarkers for myocardial infarction was assayed in the same manner as in the method for measuring CRP concentration. As can be seen from FIG. 7, assay for myoglobin at the sample line, the antigen line and the control line showed a tendency identical to the results measured for CRP, and based on these results, the same method as in CRP measurement was performed and results are shown in FIG. 8. As can be seen from FIG. 8, a complete linear result was obtained in the concentration range of 1 ng/mL to 500 μg/mL. Through myoglobin measurement results shown in FIGS. 7 and 8, it was confirmed that the measurement method according to the present invention can be applied to various antigens.

### CONCLUSION

The present inventor developed a lateral flow immuno sensor having a strip with multiple lines capable of measuring CRP over a wide range of concentrations. The sensor according to the present invention could avoid the hook effect commonly found in conventional sensors for detecting antigens of high concentrations by processing and combining signals from three immunological active lines functioning in a distinguishable manner The LFA sensor according to the present invention could complete assay of CRP concentrations ranging from 1 ng/mL to 500 μg/mL within 10 minutes. The detectable concentration was in the range of 0.67 ng/mL to 1.02 mg/mL, which is the widest range that can be measured using an LFA strip sensor (about 106 times). Since the sensor was composed of a membrane and pads that are light and relatively cheap in terms of preparation and processing, LFA systems with multiple lines are particularly advantageous in development of a device for a point of care testing (POCT). Further, the sensor according to the present invention could be easily handled and had a wide range of dynamic regions, which not only allowed CRP detection in a simple manner and with high sensitivity, but also allowed accurate diagnosis for inflammations and diseases of patients. In addition, besides CRP, in the measurement using myoglobin, it was confirmed that measurement results almost identical in level to those of CRP were obtained. Through these results, it is confirmed that the antigens applicable to the present invention are not limited to CRP, and a wide range of antigens that can be used in sandwich immunoreactions and have two antibody pairs can be employed.

Although some embodiments have been described herein, it should be understood by those skilled in the art that these embodiments are given by way of illustration only, and that various modifications, variations, and alterations can be made without departing from the spirit and scope of the invention. Therefore, the scope of the invention should be limited only by the accompanying claims and equivalents thereof.

1. A lateral flow assay strip sensor for measuring an antigen concentration over a wide range of concentrations,
wherein the lateral flow assay strip sensor comprises a structure in which a sample pad, a conjugate pad, a membrane and an absorption pad are sequentially connected,
the conjugate pad is provided with a nanoparticle-detection antibody conjugate composed of nanoparticles and a detection antibody to bind the antigen,
the membrane is provided with a test line, an antigen line and a control line formed sequentially in a direction from the conjugate pad to the absorption pad,
the test line comprises an immobilized capture antibody to bind the antigen,
the antigen line comprises an immobilized antigen,
the control line comprises an immobilized secondary antibody to bind the detection antibody.

2. The lateral flow assay strip sensor according to claim 1, wherein the antigen immobilized in the antigen line is immobilized via a capture antibody immobilized on the membrane.

3. The lateral flow assay strip sensor according to claim 1, wherein the detection antibody in the nanoparticle-detection antibody conjugate is a monoclonal antibody.

4. The lateral flow assay strip sensor according to claim 1, wherein the nanoparticle is a gold nanoparticle.

5. The lateral flow assay strip sensor according to claim 1, wherein the membrane is prepared from nitrocellulose, polyethersulfone, polyethylene, nylon, polyvinylidene fluoride, polyester, or polypropylene.

6. The lateral flow assay strip sensor according to claim 1, wherein the sample pad, the conjugate pad, the membrane and the absorption pad are sequentially connected on a backing card.

7. The lateral flow assay strip sensor according to claim 1, wherein the antigen is C-reactive protein (CRP) or myoglobin.

8. A method for measuring an antigen concentration over a wide range of concentrations using a lateral flow assay strip sensor comprising a structure in which a sample pad, a conjugate pad, a membrane and an absorption pad are sequentially connected, the conjugate pad being provided with a nanoparticle-detection antibody conjugate composed of nanoparticles and a detection antibody to bind the antigen, the membrane being provided with a test line, an antigen line and a control line formed sequentially in a direction from the conjugate pad to the absorption pad, the test line comprising an immobilized capture antibody to bind the antigen, the antigen line comprising an immobilized antigen, and the control line comprising an immobilized secondary antibody to bind the detection antibody, the method comprising:
(a) bringing a sample containing an antigen to be assayed in a predetermined concentration into contact with the sample pad;
(b) measuring signals produced from the test line, the antigen line and the control line;
(c) performing steps (a) and (b) using samples containing an antigen to be assayed in a predetermined concentration different from the concentration in step (a);
(d) calculating a calibration curve from signal values measured from steps (a) to (c);
(e) bringing a sample containing an antigen to be assayed in an unknown concentration into contact with the sample pad;
(f) measuring signals produced from the test line, the antigen line and the control line; and
(g) calculating a concentration of the antigen contained in the unknown concentration using the measured signal values and the calibration curve of step (d).

9. The method according to claim 8, wherein, in step (g), when comparing the signal values measured at the antigen line with the signal values measured at the control line, the lowest value of the calculated antigen concentrations is chosen when the signal values at the antigen line are greater than the signal values measured at the control line, and the highest value of the calculated antigen concentrations is chosen when the signal values at the antigen line are less than the signal values measured at the control line.

10. The method according to claim 8, wherein the antigen concentration capable of being assayed ranges from 1 ng/mL to 500 μg/mL.

11. The method according to claim 8, wherein the antigen immobilized at the antigen line of the lateral flow assay strip sensor is immobilized via a capture antibody immobilized on the membrane.

12. The method according to claim 8, wherein the detection antibody in the nanoparticle-detection antibody conjugate of the lateral flow assay strip sensor is a monoclonal antibody.
